(19) United States
(12) Patent Application Publication
(10) Pub. No.: US 2005/0198701 A1
(43) Pub. Date: Sep. 8, 2005

(54) Method of generating new fluorescent fish by breeding transgenic fluorescent fish with fish with different phenotype and new fluorescent fish generated therefrom

(76) Inventors: Lin Shiue Lian, Taipei (TW); Chen Yin Chun, Taipei (TW)

Correspondence Address: HARNESS, DICKEY & PIERCE, P.L.C., P.O. BOX 8910, RESTON, VA, 20195, US

(21) Appl. No.: 10/791,536

(22) Filed: Mar. 3, 2004

(51) Int. Cl. A01K 67/027

(52) U.S. Cl. 800/20

(57) ABSTRACT

The present invention relates to a method of generating new ornamental fish by breeding transgenic fish with fish with different phenotype or behavior pattern. The resulting transgenic fish shows different phenotype and behavior pattern from each of its parents. The present invention also provides the new ornamental fish generated from the method.

### FIELD OF THE INVENTION

The invention relates to a method of generating new ornamental fish and the new ornamental fish generated therefrom.

### DESCRIPTION OF THE PRIOR ART

Transgenic technology has become an important tool for the study of gene function. The technique was first developed in mice. They injected foreign DNA into fertilized eggs and found that some of the mice developed from the injected eggs retained the foreign DNA.

Fish are also an intensive research subject of transgenic studies. There are many ways of introducing a foreign gene into fish, including: microinjection, electroporation, sperm-mediated gene transfer, gene bombardment or gene gun, liposome-mediated gene transfer and the direct injection of DNA into muscle tissue (Xu et al., DNA Cell Biol. 18,85-95 (1999)).

Transgenic fish has also been generated for the entertainment purpose. For example, transgenic fish expressing GFP fluorescence by introduction of a GFP gene fused with a fish-specific gene promoter into fertilized eggs, has been generated using medaka. [Hamada, K. et al., Mol. Marine Biol. Biotech., 7, 173-180 (1998)]. Tanaka et al produced a transgenic medaka with green fluorescence in only germ cells of himedaka [in The 22nd Annual Meeting of the Molecular Biology Society of Japan, Program, Abstract, pp. 458 (1999), Tanaka, et al]. Using transgenic technique to generate new ornamental fish that exhibits different fluorescent colors or phenotypic patterns one by one. However, the technique is apparently expensive, labor intensive and time exhausting.

Fish breeding has the objective to produce improved fish varieties based on the exploitation of genetic variation. The genotype of a progeny fish is the result of the combination of the genotypes of the male and female gamete, which through fusion resulted in a zygote, from which ultimately the progeny fish developed. Therefore, genetic breeding between transgenic fish with fish of the same or different species with different phenotypes or behavior patterns, such as fish with different spot patterns, stripe colors, or even susceptibility to the environmental changes, provided us a much greater choices of ornamental fish that could never been achieved before the arrival of transgenic fish and these fish can be generated in a much faster and less expensive way than performing transgenic techniques each and every time.

### SUMMARY OF THE INVENTION

The present invention provides a method of generating new ornamental fish, comprising steps of (a) generating a transgenic fish containing one or more fused fluorescent gene; (b) breeding the transgenic fish with fish with different phenotype or pattern; and (c) screening for new transgenic progenies showing different phenotype or pattern from their parents.

The present invention also provides the new transgenic fish generated from the method.

### BRIEF DESCRIPTION OF THE DRAWING

FIG. 1 illustrates the plasmid construct (a) pβ-actin-EGFP; (b) pβ-actin-EGFP-ITR.

FIG. 2 illustrates the construction of plasmid pβ-DsRed2-1-ITR from pOBA-109 and pDsRed2-1-ITR.

FIG. 3 illustrates the plasmid construct, p-αDsRedITR, with the inserted gene fragment and restriction sites.

FIG. 4 shows the breeding between red TK-1 with Oryzias curvinotus, green TK-1 with Oryzias curvinotus, red TK-2 with Brachydanio sp, red TK-2 with Brachydanio frankei, and red TK-2 with Brachydanio rerio.

FIG. 5 shows the picture of (a) red TK-1, (b) Oryzias curvinotus and (c) red TK-1×Oryzias curvinotus.

FIG. 6 shows the picture of (a) green TK-1, (b) Oryzias curvinotus and (c) green TK-1×Oryzias curvinotus.

FIG. 7 shows the picture of (a) red TK-2, (b) Brachydanio sp and (c) red TK-2×Brachydanio sp.

FIG. 8 shows the picture of (a) red TK-2, (b) Brachydanio frankei and (c) red TK-2×Brachydanio frankei.

FIG. 9 shows the picture of (a) red TK-2, (b) Brachydanio rerio and (c) Purple Zebra Fish.

### DETAIL DESCRIPTION OF THE INVENTION

The present invention provides a method of generating new ornamental fish, which exhibit different phenotype or pattern from its parent, and the new transgenic fish generated by the method.

As used herein, ornamental fish refers to transgenic fish, or progeny of a fish, into which an exogenous construct has been introduced. A fish into which a construct has been introduced includes fish that have developed from embryonic cells into which the construct has been introduced. As used herein, an exogenous construct is a nucleic acid that is artificially introduced, or was originally artificially introduced, into a fish. The term artificial introduction is intended to exclude introduction of a construct through normal reproduction or genetic crosses. That is, the original introduction of a gene or trait into a line or strain of animal by cross breeding is intended to be excluded. However, fish produced by transfer, through normal breeding, of an exogenous construct (that is, a construct that was originally artificially introduced) from a fish containing the construct are considered to contain an exogenous construct. Such fish are progeny of fish into which the exogenous construct has been introduced. As used herein, progeny of a fish are any fish which are descended from the fish by sexual reproduction or cloning, and from which genetic material has been inherited. In this context, cloning refers to production of a genetically identical fish from DNA, a cell, or cells of the fish. The fish from which another fish is descended is referred to as a progenitor fish. As used herein, development of a fish from a cell or cells (embryonic cells, for example), or development of a cell or cells into a fish, refers to the developmental process by which fertilized egg cells or embryonic cells (and their progeny) grow, divide, and differentiate to form an adult fish.

The examples illustrate the manner in which new transgenic fish exhibiting different phenotype or patterns from its progenitor fish can be made and used. The transgenic fish described in the examples are particularly useful to increase the commercial value of ornamental fish.

Transgene Construct

Transgene constructs are the genetic material that is introduced into fish to produce a transgenic fish. Such constructs are artificially introduced into fish. The manner of introduction, and, often, the structure of a transgene construct, render such a transgene construct an exogenous construct. Although a transgene construct can be made up of any nucleic acid sequences, for use in the disclosed transgenic fish it is preferred that the transgene constructs combine expression sequences operably linked to a sequence encoding an expression product. The transgenic construct will also preferably include other components that aid expression, stability or integration of the construct into the genome of a fish. As used herein, components of a transgene construct referred to as being operably linked or operatively linked refer to components being so connected as to allow them to function together for their intended purpose.

Fish

The disclosed constructs and methods can be used with any type of fish. As used herein, fish refers to any member of the classes collectively referred to as pisces. It is preferred that fish belonging to species and varieties of fish of commercial or scientific interest be used. These fish include Cichlidae (such as Pseudotropheus, Cichlasoma, Apistogramma, Pterophyllum or Symohysodon), Fighting fish (such as Betta or Macropodus), Catfish (such as Corydoras, Ancistrus or Pterygoplichthys), Characidae (such as Tetras or Carnegiella), Cyprinidae (such as Cyprinus, Brachydanio (zebrafish), Danio or Carassius) and Killifish (such as Medaka, Rivulines or Livebearing Toothcarps).

The preferred fish for use with the disclosed constructs and methods is medaka and zebrafish (Brachydanio or Danio). Medaka and zebrafish have significant advantages to use for ornamental fish that they are largely transparent (Kimmel, Trends Genet 5:283-8 (1989)). General zebrafish care and maintenance is described by Streisinger, Natl. Cancer Inst. Monogr. 65:53-58 (1984). The medaka is selected from the group consisting of Oryzias javanicus, Oryzias latipes, Oryzias nigrimas, Oryzias luzonensis, Oryzias profundicola, Oryzias matanensis, Oryzias mekongensis, Oryzias minutillus, Oryzias melastigma, O. curvinotus, O. celebensis. X. oophorus, and X. saracinorum. The most preferred is Oryzias latipes.

The preferred Brachydanio is selected from the group consisting of D. acrostomus, D. aequipinnatus, D. malabaricus, D. albolineatus, D. annandalei, D. apogon, D. apopyris, D. assamensis, D. choprae, D. chrysotaeniatus, D. dangila, D. devario, D. fangfangae, D. frankei, D. fraseri, D. gibber, D. interruptus, D. kakhienensis, D. kyathit, D. laoensis, D. leptos, D. maetaengensis, D. malabaricus, D. naganensis, D. neilgherriensis, D. nigrofasciatus, D. pathirana, D. regina, D. rerio, D. roseus, D. salmonata, D. shanensis and D. spinosus. The most preferred is D. rerio.

Medaka and zebrafish embryos are easily accessible and nearly transparent. Given these characteristics, a transgenic medaka or zebrafish egg or embryo, carrying a construct encoding a reporter protein and systemic expression sequences, can provide a rapid real time in vivo identification of successful transgenics. Other fish with some or all of the same desirable characteristics are also preferred.

Generation of the Progenitor Transgenic Fish

The disclosed transgenic fish are produced by introducing a transgene construct into cells of a fish, preferably embryonic cells, and most preferably in a single cell embryo. Where the transgene construct is introduced into embryonic cells, the transgenic fish is obtained by allowing the embryonic cell or cells to develop into a fish. Introduction of constructs into embryonic cells of fish, and subsequent development of the fish, are simplified by the fact that embryos develop outside of the parent fish in most fish species.

The disclosed transgene constructs can be introduced into embryonic fish cells using any suitable technique. Many techniques for such introduction of exogenous genetic material have been demonstrated in fish and other animals. These include microinjection, electroporation, particle gun bombardment, and the use of liposomes. The preferred method for introduction of transgene constructs into fish embryonic cells is by microinjection.

Embryos or embryonic cells can generally be obtained by collecting eggs immediately after they are laid. Depending on the type of fish, it is generally preferred that the eggs be fertilized prior to or at the time of collection. This is preferably accomplished by placing a male and female fish together in a tank that allows egg collection under conditions that stimulate mating. After collecting eggs, it is preferred that the embryo be exposed for introduction of genetic material by removing the chorion. This can be done manually or, preferably, by using a protease such as pronase. A fertilized egg cell prior to the first cell division is considered a one cell embryo, and the fertilized egg cell is thus considered an embryonic cell.

After introduction of the transgene construct the embryo is allowed to develop into a fish. This generally need involve no more than incubating the embryos under the same conditions used for incubation of eggs. However, the embryonic cells can also be incubated briefly in an isotonic buffer. If appropriate, expression of an introduced transgene construct can be observed during development of the embryo.

Fish harboring a transgene can be identified by any suitable means. For example, the genome of potential transgenic fish can be probed for the presence of construct sequences. To identify transgenic fish actually expressing the transgene, the presence of an expression product can be assayed. Several techniques for such identification are known and used for transgenic animals and most can be applied to transgenic fish. Probing of potential or actual transgenic fish for nucleic acid sequences present in or characteristic of a transgene construct is preferably accomplished by Southern or Northern blotting. Also preferred is detection using polymerase chain reaction (PCR) or other sequence-specific nucleic acid amplification techniques. Transgenic medaka and zebrafish that are fluorescence because of the transgene they carry can be directly observed with unaided eye and are described in the examples.

Generating the New Transgenic Fish

The invention relates to a process for the production of haploid cells and to the production of the new transgenic fish from these cells.

According to the invention haploid cells are preferably derived from a meiotic process. The process of meiosis forms the pivotal event in the life cycle of living organisms at which genetic variation is created. Moreover it marks the transition between the diploid and the haploid. The specialized cell in the female reproductive organ that enters meiosis, which is called oocyte cell, is embedded in the differentiated ovule inside the ovary. The oocytes go through meiosis and generate haploid eggs.

Within the male reproductive tissues, a similar process leads to the formation of haploid sperms.

Although there are significant differences in the cellular processes leading to the formation of female and male gametes, the cytological events that occur during female and male meiosis are very similar suggesting the involvement of common gene products.

The end product of meiosis is a set of four genetically distinct haploid cells, which can undergo mitosis to develop into gametes. The gamete, which upon fusion leads to the formation of a zygote, develops into an embryo that can grow out the next generation.

In order to generate the new transgenic fish, the progenitor fish carrying the transgene are bred with the other progenitor fish with different phenotype or patterns. The phenotype of the fish is selected from the group consisting of colors, fluorescent colors, body shapes, body sizes, body transparent levels, grain colors, stripe colors, fin shapes, fin sizes, tail shapes, tail sizes, eye colors, eye shapes; and any observable phenotypic differences from those of fluorescent mate.

The pattern of the fish is selected from the group consisting of grain patterns, stripe patterns and swimming patterns.

Haploid gametes carrying the transgene from the progenitor fish were fertilized with haploid gametes from the other progenitor fish with different phenotypes of patterns. The fertilization can be done using any suitable breeding techniques available. Specie-specific breeding condition may be required. Example of generating new transgenic medaka and zebrafish is described in the examples.

The transgenic fish and the fish with different phenotype or pattern may be of the same or different family, genus or species.

The preferred fish for use with the disclosed methods is medaka and zebrafish (Brachydanio or Danio). The medaka is selected from the group consisting of Oryzias javanicus, Oryzias latipes, Oryzias nigrimas, Oryzias luzonensis, Oryzias profundicola, Oryzias matanensis, Oryzias mekongensis, Oryzias minutillus, Oryzias melastigma, O. curvinotus, O. celebensis, X. oophorus, and X. saracinorum. The most preferred is Oryzias latipes.

The preferred Brachydanio is selected from the group consisting of D. acrostomus, D. aequipinnatus, D. malabaricus, D. albolineatus, D. annandalei, D. apogon, D. apopyris, D. assamensis, D. choprae, D. chrysotaeniatus, D. dangila, D. devario, D. fangfangae, D. frankei, D. fraseri, D. gibber, D. interruptus, D. kakhienensis, D. kyathit, D. Iaoensis, D. leptos, D. maetaengensis, D. malabaricus, D. naganensis, D. neilgherriensis, D. nigrofasciatus, D. pathirana, D. regina, D. rerio, D. roseus, D. salmonata, D. shanensis and D. spinosus. The most preferred is D. rerio.

Those skilled in the art will recognize, or be able to ascertain using no more than routine experimentation, many equivalents to the specific embodiments of the invention described herein. Such equivalents are intended to be encompassed by the following claims.

### EXAMPLES

The examples below are non-limiting and are merely representative of various aspects and features of the present invention.

### Example 1

### Generation of the Plasmid pβ-actin-EGFPITR

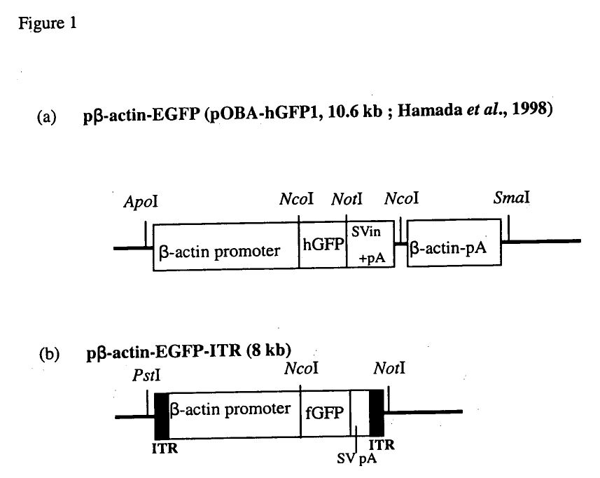

In order to generate the green fluorescent medaka—green TK-1, plasmid pβ-actin-EGFPITR was constructed. The plasmid of the invention was described in Chou CY et al, Transgenic Res. 2001 10(4):303-15, and are presented in FIG. 1. The pβ-actin-EGFP contained a medaka β-actin promoter fused with hGFP1 cDNA, an intron of small t antigen, SV40 polyA, and polyA from the medaka β-actin gene. The medaka β-actin promoter was obtained from pOBA-hGFP1 and was digested with SalI and NcoI. The 3.8 kb fragment end-product was ligated to the 4.2 kb SalI-NcoI fragment from pCMV-EGFPITR. The final result was an 8 kb pβ-actin-EGFPITR plasmid in which EGFP cDNA was driven by the β-actin promoter and flanked at both ends by AAV-ITR.

Appropriate amount of pβ-actin-EGFPITR was restricted by NotI. The molecular mass for the pβ-actin-EGFPITR DNA fragment was 7.6 kb as expected.

### Example 2

### Generation of the Plasmid pβ-DsRed2-1-ITR

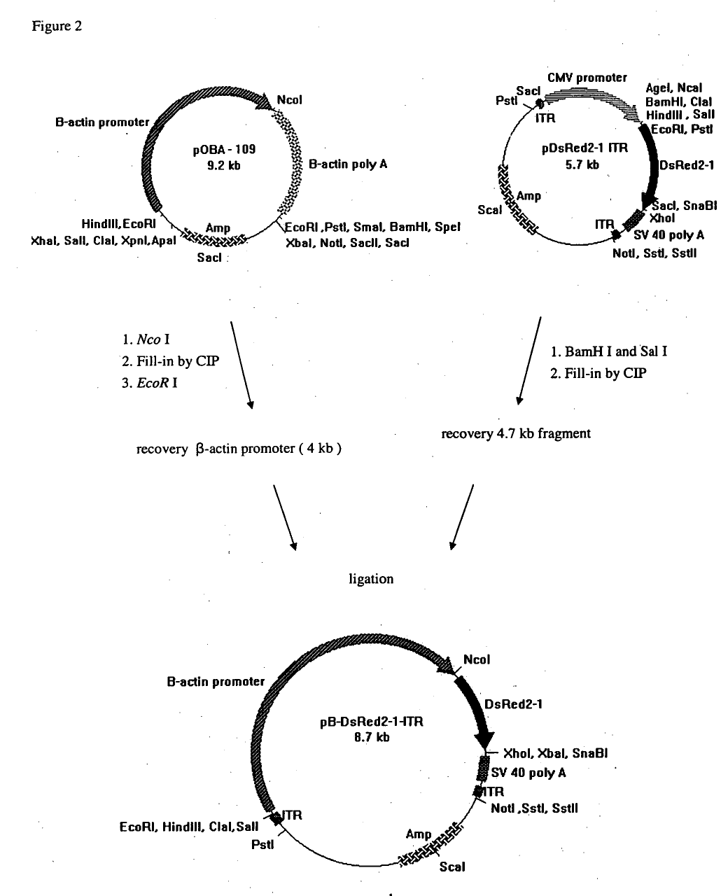

In order to generate the red fluorescent medaka—red TK-1, plasmid pβ-DsRed2-1-ITR was constructed. As illustrated in FIG. 2, the medaka β-actin gene promoter was obtained by digesting plasmid construct pOBA-109 with restriction enzymes NcoI and EcoRI. NcoI was used first, ends were filled in, and a subsequent digestion with EcoRI provided a 4 kb fragment.

As illustrated in FIG. 2, the CMV promoter was cut out by digesting the construct pDsRed2-1-ITR with restriction enzymes BamHI and SalI. Digestion with BamHI and SalI provided a 4.7 kb fragment. Then, the β-actin gene promoter of medaka was inserted into the plasmid construct, pDsRed2-1-ITR, at the position where the CMV promoter was cut out. The resulting plasmid construct had two 145 bp adeno-associated virus inverted terminal repeats (ITR). One ITR was located at the 3′ end of SV40 poly A. The other was located at the 5′ end of the β-actin gene promoter.

As illustrated in FIG. 2, the resulting plasmid construct, pβ-DsRed2-1-ITR, had a total length of 8.7 kb. pβ-DsRed2-1-ITR contained (1) the medaka β-actin gene promoter (for ubiquitous expression of whole body); (2) sea coral red fluorescent protein; (3) adeno-associated virus inverted terminal repeats; and (4) pUC plasmid construct basis.

Appropriate amount of pβ-DsRed2-1-ITR was digested with proportional amount of Not I restriction enzyme. A small fraction of the digested product was analyzed by agarose gel electrophoresis to verify its linearity. The fragment length was 8.7 kb as expected.

### Example 3

### Generation of Plasmid p-αDsRedITR

In order to generate the red fluorescent zebrafish—red TK-2, plasmid β-αDsRed2-1-ITR was constructed. As illustrated in FIG. 1, the golden zebrafish α-actin gene promoter was obtained by digesting plasmid construct p-αEGFPITR with restriction enzymes NcoI and SalI. NcoI was used first, ends were filled in, and a subsequent digestion with SalI provided a 3.8 kb fragment.

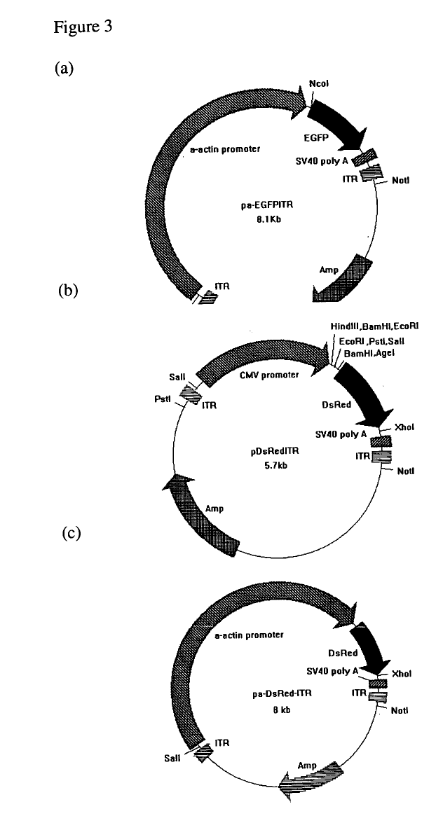

As illustrated in FIG. 3, the CMV promoter was cut out by digesting the construct pDsRedITR with restriction enzymes BamHI and SalI. BamHI was used first, ends were filled in, and a subsequent digestion with SalI provided a 4.2 kb fragment. Then, the α-actin gene promoter of golden zebrafish was inserted into the plasmid construct, pDsRedITR, at the position where the CMV promoter was cut out. The resulting plasmid construct had two 145 bp adeno-associated virus inverted terminal repeats (ITR). One ITR was located at the 3′ end of SV40 poly A. The other was located at the 5′ end of the α-actin gene promoter.

As illustrated in FIG. 3, the resulting plasmid construct, p-αDsRedITR, had a total length of 8.0 kb. The resulting construct p-αDsRedITR contained (1) the golden zebrafish α-actin gene promoter (for systemic gene expression); (2) sea coral red fluorescent protein; (3) adeno-associated virus inverted terminal repeats; and (4) pUC plasmid construct basis.

Appropriate amount of p-αDsRedITR was digested with proportional amount of Not I restriction enzyme. A small fraction of the digested product was analyzed by agarose gel electrophoresis to verify its linearity. The fragment length was 8 kb as expected.

### Example 4

### Preparation of Microinjected DNA

All DNA plasmids were prepared via ultra-centrifugation with cesium chloride and ethidium bromide gradient (Radloff et al., 1967 Proc Natl Acad Sci USA 57:1514-1521). All DNA fragments used for microinjection were eluted from agarose gel following electrophoresis.

### Example 5

### Cytoplasmic Microinjection

The procedures followed for cytoplasmic microinjection are described in detail in Kinoshita and Ozato, 1995 Fish Biol J MEDAKA 7:59-64 and Kinoshita et al. 1996 Aquaculture 143:267-276.

Fish were maintained under artificial conditions of 14 h light and 10 h darkness at 26° C. and maintained on a diet of Tetramin (Tetra, Germany). Before the incubator entered the dark cycle, fish were collected and separated by separation net. On the next morning after the light cycle has begun, fish eggs were collected every 15-20 minutes.

Eggs were collected within 30 min of fertilization and attaching filaments removed. The linearized construct was quantified and dissolved in 5×PBS with phenol red at the desired concentration. DNA was picked up by micro-capillary of zebrafish microinjector (Drummond) wherein the injection needle width of the micro-capillary was approximately 10 μm. As micro-needle enters the cell cytoplasm, the DNA injected was approximated 2-3 nl. In each microinjection session, 30-40 eggs were injected; 250-300 eggs were injected in each experiment. Injected eggs were incubated at 26° C. in distilled water.

### Example 6

### Hatching and Screening for Transgenic Embryos

Injected eggs were rinsed with sterilized solution, cultured in incubator wherein the temperature was 28.5° C. The fluorescence could be observed in the developing embryo after 24 hours.

Embryos were observed under a bright field with a dissecting stereomicroscope (MZAPO, Leica, Germany). Dark field illumination for detecting green fluorescence was performed with a stereomicroscope equipped with a GFP Plus filter (480 nm). The distribution and intensity of the red fluorescence is observed under fluorescence microscope (Leica MZ-12; Fluorescence System: light source Hg 100 W; main emission wavelength 558 nm, and main absorption wavelength 583 nm, filter set RFP-Plus; photography system MPS60). Photographs were taken using an MPS60 camera loaded with ISO 400 film and equipped with a controller for film exposure time (Leica, Germany). In order to examine the distribution of GFP or RFP expression in the tissues of transgenic fish, 11 d post-fertilization larva which having GFP or RFP expression on appearance were sectioned and observed under fluorescent microscopy. Larva were fixed for 30 min in 4% paraformaldehyde at 4° C., embedded in cryomatrix (Shandon, USA) and frozen at −20° C. Cryostat sections (Cryostat Microtome, HM500 OM, Microm, Germany) with 15 μm thickness were mount on slides and observed the GFP or RFP fluorescence immediately.

### Example 7

### Generation of New Transgenics

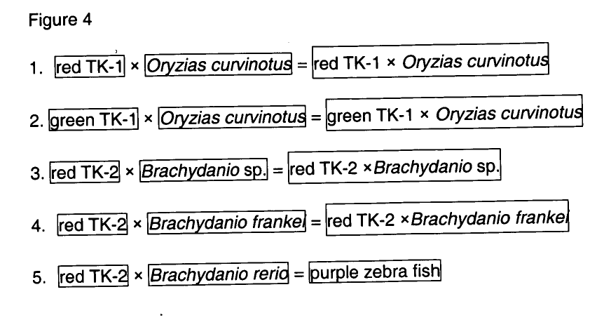

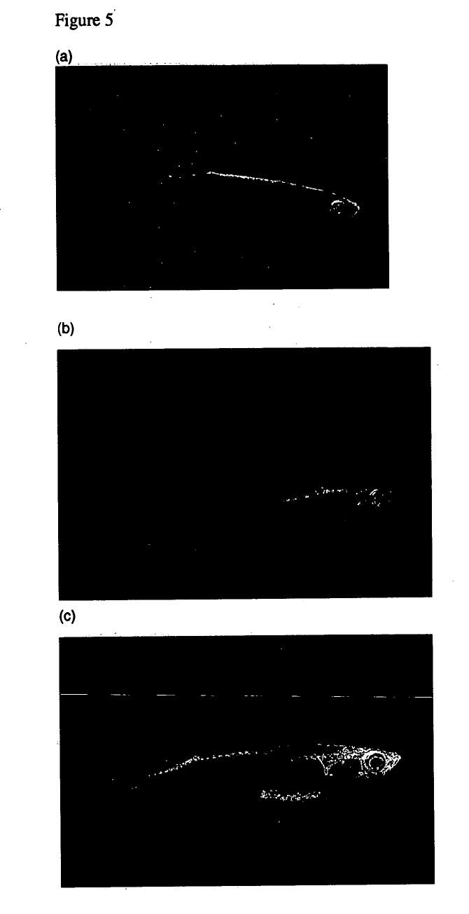

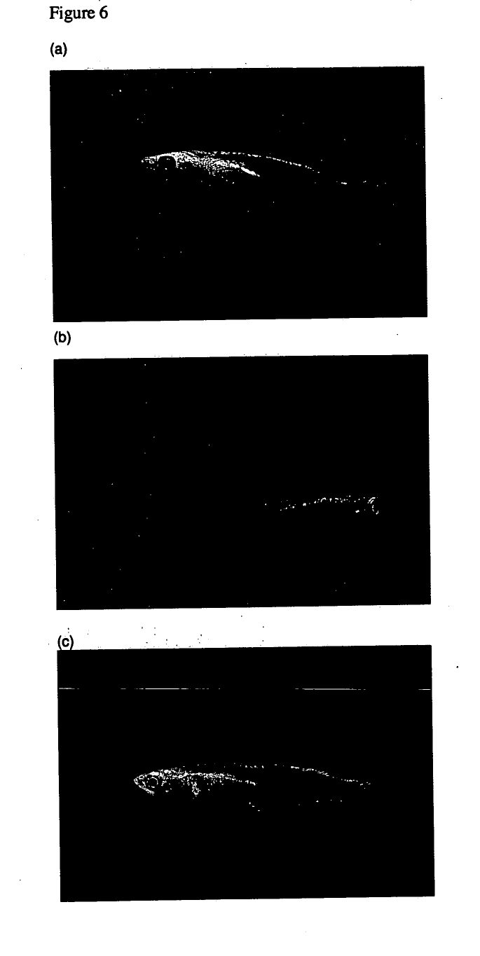

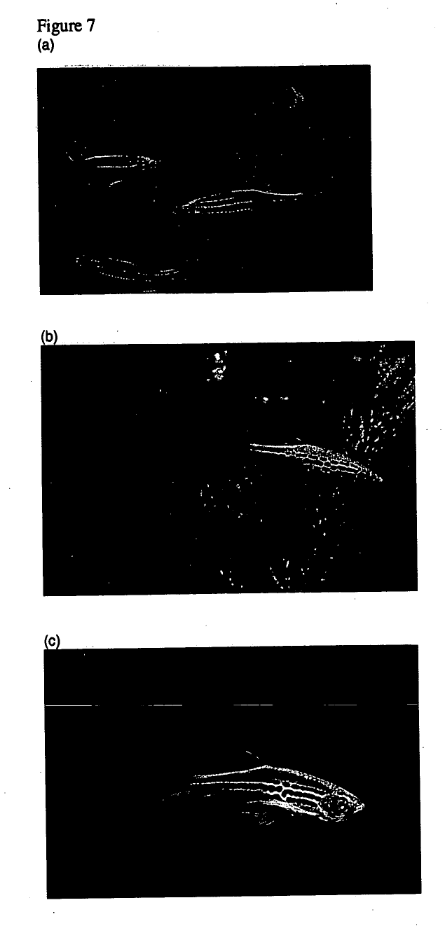

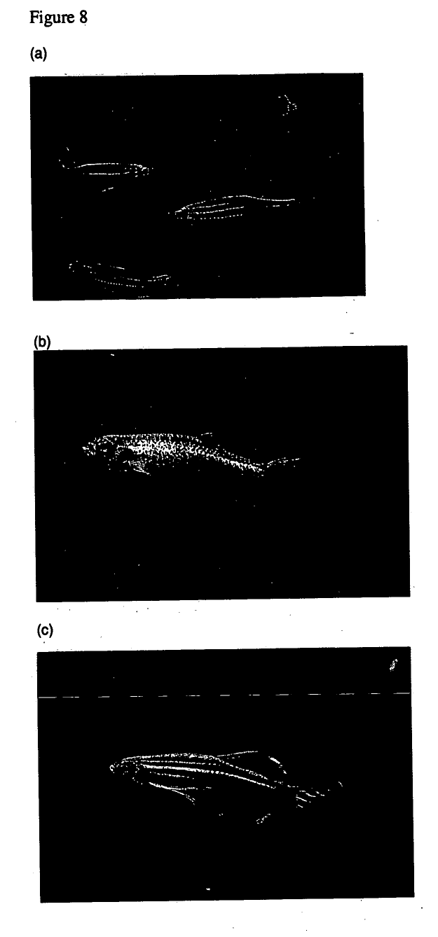

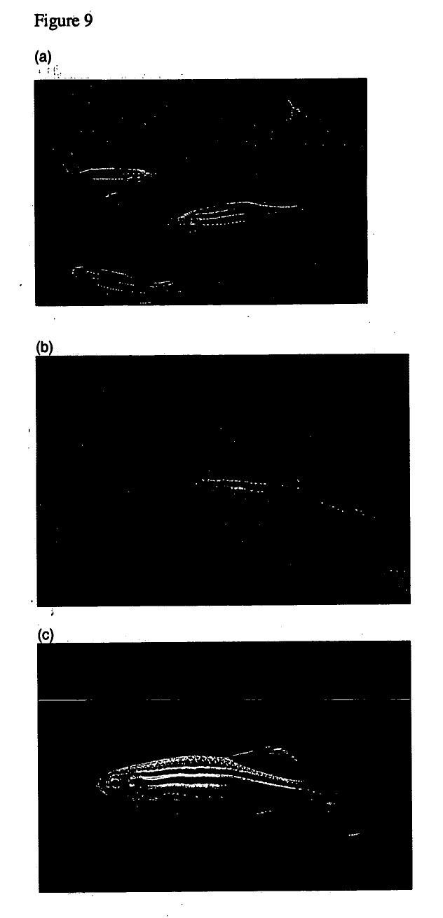

As shown in FIG. 4, genetic breeding was performed between red TK-1 (FIG. 5 (a)) with Oryzias curvinotus (FIG. 5 (b)), green TK-1 (FIG. 6 (a)) with Oryzias curvinotus (FIG. 6 (b)), red TK-2 (FIG. 7 (a)) with Brachydanio sp (FIG. 7 (b)), red TK-2 (FIG. 8 (a)) with Brachydanio frankei (FIG. 8 (b)), red TK-2 (FIG. 9 (a)) with Brachydanio rerio (FIG. 9 (b)).

The resulting F1 progeny transgenics, namely red TK-1×Oryzias curvinotus (FIG. 5 (c)), green TK-1×Oryzias curvinotus (FIG. 6 (c)), red TK-2×Brachydanio sp (FIG. 7 (c)), red TK-2×Brachydanio frankei (FIG. 8 (c)), Purple Zebra Fish (FIG. 9 (c)), are products of the genetic varieties of their parents. In these examples, one of the progenitor fish exhibited green or red fluorescence whereas the other showed a specific stripe or grain pattern. The new trasngenics generated from the breeding thus inherited both characteristics and showed phenotypes that are different from each of the parent fish.

These new transgenics are self-crossed to generate F2 homozygotes and hatched as described in example 6. All of the progenies of the F2 homozygotes will inherit the transgene and show the new phenotypes.

While the invention has been described and exemplified in sufficient detail for those skilled in this art to produce and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cell lines, embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

1. A method for generating an ornamental fish, comprising steps of (a) generating a transgenic fish containing one or more fused fluorescent gene; (b) breeding the transgenic fish with fish with different phenotype or pattern; and (c) screening the new transgenic progenies showing different phenotype or pattern from their parents.

2. The method as set forth in claim 1, wherein the transgenic fish and the fish with different phenotype or pattern are the same or different family, genus or species.

3. The method as set forth in claim 2, wherein the transgenic fish and the fish with different phenotype or pattern are different genus or species.

4. The method as set forth in claim 3, wherein the transgenic fish and the fish with different phenotypes or patterns are different species.

5. The method as set forth in claim 4, wherein the fluorescent gene is selected from the group consisting of green fluorescent protein (GFP), modified green fluorescent protein, enhanced green fluorescent protein (EGFP), red fluorescent protein (RFP), enhanced red fluorescent protein (ERFP), blue fluorescent protein (BFP), enhanced blue fluorescent protein (EBFP), yellow fluorescent protein (YFP), enhanced yellow fluorescent protein (EYFP), cyan fluorescent protein (CFP), and enhanced cyan fluorescent protein (ECFP).

6. The method as set forth in claim 5, wherein the fluorescent gene is selected from the group consisting of green fluorescent protein (GFP), modified green fluorescent protein, enhanced green fluorescent protein (EGFP), red fluorescent protein (RFP), enhanced red fluorescent protein (ERFP), blue fluorescent protein (BFP) and enhanced blue fluorescent protein (EBFP).

7. The method as set forth in claim 6, wherein the fluorescent gene is selected from the group consisting of green fluorescent protein (GFP), modified green fluorescent protein, enhanced green fluorescent protein (EGFP), red fluorescent protein (RFP) and enhanced red fluorescent protein (ERFP).

8. The method as set forth in claim 1, wherein the phenotype of the fish is selected from the group consisting of colors, body shapes, body sizes, body transparent levels, grain colors, stripe colors, fin shapes, fin sizes, tail shape, tail sizes, eye color, eye shapes; and any observable phenotypic differences from those of fluorescent mate.

9. The method as set forth in claim 8, wherein the phenotype of the fish is selected from the group consisting of colors, body shapes, body transparent levels, grain colors and stripe colors.

10. The method as set forth in claim 1, wherein the pattern of the fish is selected from the group consisting of grain patterns, stripe patterns and swimming patterns.

11. The method as set forth in claim 4, wherein the fish is selected from the group consisting of Cichlidae, Fighting fish, Catfish, Characidae, Cyprinidae and Killifish.

12. The method as set forth in claim 11, wherein the Cichlidae is Pseudotropheus, Cichlasoma, Apistogramma, Pterophyllum or Symohysodon.

13. The method as set forth in claim 11, wherein the Fighting fish is Betta or Macropodus.

14. The method as set forth in claim 11, wherein the Catfish is Corydoras, Ancistrus or Pterygoplichthys.

15. The method as set forth in claim 11, wherein the Characidae is Tetras or Carnegiellaor.

16. The method as set forth in claim 11, wherein the Cyprinidae is Cyprinus, Brachydanio(zebrafish), Danio or Carassius.

17. The method as set forth in claim 11, wherein the Killifish is Medaka, Rivulines or Livebearing Toothcarps.

18. The method as set forth in claim 16, wherein the zebrafish is selected from the group consisting of D. acrostomus, D. aequipinnatus, D. malabaricus, D. albolineatus, D. annandalei, D. apogon, D. apopyris , D. assamensis, D. choprae, D. chrysotaeniatus, D. dangila, D. devario, D. fangfangae, D. frankei, D. fraseri, D. gibber, D. interruptus, D. kakhienensis, D. kyathit, D. laoensis, D. leptos, D. maetaengensis, D. malabaricus, D. naganensis, D. neilgherriensis, D. nigrofasciatus, D. pathirana, D. regina, D. rerio, D. roseus, D. salmonata, D. shanensis D. spinosus, Brachydanio frankei and Branchydanio sp.

19. The method as set forth in claim 17, wherein the medaka is selected from the group consisting of Oryzias javanicus, Oryzias latipes, Oryzias nigrimas, Oryzias luzonensis, Oryzias profundicola, Oryzias matanensis, Oryzias mekongensis, Oryzias minutillus, Oryzias melastigma, O. curvinotus, O. celebensis. X. oophorus, and X. saracinorum.

20. The method as set forth in claim 1, wherein the new transgenic progenies is selected from the group consisting of Cichlasoma, TK1 red×O. curvinotus, TK1 green×O. curvinotus, TK2 red×Brachydanio frankei, TK2 red×Branchydanio sp, TK2 green×Brachydanio frankei, TK2 green×Branchydanio sp and Purple Zebra Fish.

21. The method as set forth in claim 20, wherein the new transgenic progenies is TK1 red×O. curvinotus, TK1 green×O. curvinotus, TK2 red×Brachydanio frankei, TK2 red×Branchydanio sp and Purple Zebra Fish.

22. The method as set forth in claim 21, wherein the new transgenic progenies is TK2 red×Brachydanio frankei or TK2 red×Branchydanio sp.

23. The ornamental fish which is prepared according to claim 1.

24. The fish as set forth in claim 23 which is TK1 red×O. curvinotus, TK1 green×O. curvinotus, TK2 red×Brachydanio frankei, TK2 red×Branchydanio sp or Purple Zebra Fish.
